(19) United States
(12) Patent Application Publication
(10) Pub. No.: US 2016/0345746 A1
(43) Pub. Date: Dec. 1, 2016

(54) Methods, Systems, and Computer Program Products For Tracking Operation of A Comfort Product

(71) Applicant: L&P Property Management Company, South Gate, CA (US)

(72) Inventors: Terrance L. Myers, Joplin, MO (US); John A. Garrett, Carthage, MO (US); Jason B. Turner, Joplin, MO (US); Michael D. Miller, Webb City, MO (US); Daniel J. Hawn, Pittsburg, KS (US); Caleb Browning, Carthage, MO (US)

(21) Appl. No.: 15/164,377

(22) Filed: May 25, 2016

Related U.S. Application Data
(60) Provisional application No. 62/168,129, filed on May 29, 2015.

(51) Int. Cl. A47C 31/00; A61B 5/11; A61B 5/00

(52) U.S. Cl. CPC A47C 31/00; A61B 5/4812; A61B 5/1115; A61B 5/6892; A61B 5/7445

(57) ABSTRACT

Methods, systems, and computer program products for tracking operation of a comfort product include determining an occupancy record for the comfort product. An indication of whether an action relating to the comfort product is recommended is provided based on the occupancy record.

This application claims the benefit of U.S. Provisional Patent Application Ser. No. 62/168,129 filed May 29, 2015, which is fully incorporated by reference herein.

### TECHNICAL FIELD

The present invention generally relates to comfort products and, more particularly, to methods, systems, and computer program products for tracking operation of a comfort product.

### BACKGROUND

Comfort products, such as a mattress, have a limited useful lifespan. In particular, a mattress develops patterns of wear over time that deteriorate the mattress's quality and comfort. The rate at which these patterns develop generally depends on the frequency of use of the mattress and whether the mattress receives proper care. For example, mattress manufacturers typically recommend that a mattress be flipped and rotated at regular intervals, such as every three months, to promote even distribution of wear and thereby extend the life of the mattress. Mattress manufacturers further recommend that a mattress be replaced about every ten years. Even with proper care, a mattress eventually reaches the end of its recommended lifespan, at which time use of the mattress may promote poor sleep habits and cause injury, such as back injury.

A need therefore exists for improved methods, systems, and computer program products for tracking operation of a comfort product, such as a mattress, to help ensure that the comfort product receives proper care and is not utilized past its recommended lifespan.

### SUMMARY

According to one aspect of the present invention, a method for tracking operation of a mattress includes determining, by at least one processor, an occupancy record for the mattress. The method further includes providing, by the at least one processor, an indication of whether an action relating to the mattress is recommended based on the occupancy record.

In another aspect, a system for tracking operation of a mattress includes at least one processor and a memory. The memory stores instructions that, when executed by the at least one processor, cause the system to determine an occupancy record for the mattress and provide an indication of whether an action relating to the mattress is recommended based on the occupancy record.

In another aspect, a device for tracking operation of a mattress includes a housing defining an interior, at least one processor positioned in the interior of the housing, and a memory positioned in the interior of the housing. The memory stores instructions that, when executed by the at least one processor, cause the device to determine an occupancy record for the mattress and provide an indication of whether an action relating to the mattress is recommended based on the occupancy record.

### BRIEF DESCRIPTION OF THE DRAWINGS

The accompanying drawings, which are incorporated in and constitute a part of this specification, illustrate various embodiments of the invention and, together with the general description of the invention given above, and the detailed description of the embodiments given below, serve to explain the embodiments of the invention.

FIG. 1 is a schematic view of an exemplary operating environment that includes a device for tracking operation of a comfort product.

FIG. 2 is a schematic view of an exemplary computer system that may be utilized in the operating environment of FIG. 1.

FIG. 3 is an isometric view of an exemplary mattress that includes a device for tracking the operation of the mattress.

FIG. 4 is a schematic view of an exemplary surface of the tracking device of FIG. 1 that includes a visual indicator.

FIG. 5 is a schematic view of an exemplary interaction between a mattress that includes the tracking device of FIG. 1 and a foundation adapted to support the mattress.

FIG. 6 is a schematic view of an exemplary mattress tracking system that may be provided by the tracking device of FIG. 1.

FIG. 7 is a flowchart of an exemplary process for tracking the operation of a mattress that may be performed by the mattress tracking system of FIG. 6.

FIG. 8 is a flowchart of an exemplary process for determining an occupancy record for a mattress that may be performed by the mattress tracking system of FIG. 6.

FIG. 9 is a flowchart of an exemplary process for determining whether a presence detected on a mattress is related to a sleep cycle that may be performed by the mattress tracking system of FIG. 6.

FIG. 10 is a flowchart of an exemplary process for providing an indication of whether an action relating to a mattress is recommended based on an occupancy record that may be performed by the mattress tracking system of FIG. 6.

FIG. 11 is a flowchart of another exemplary process for providing an indication of whether an action relating to a mattress is recommended based on an occupancy record that may be performed by the mattress tracking system of FIG. 6.

FIG. 12 is a flowchart of an exemplary process for tracking the operation of a mattress using a remote computing device that may be performed by the mattress tracking system of FIG. 6.

### DETAILED DESCRIPTION

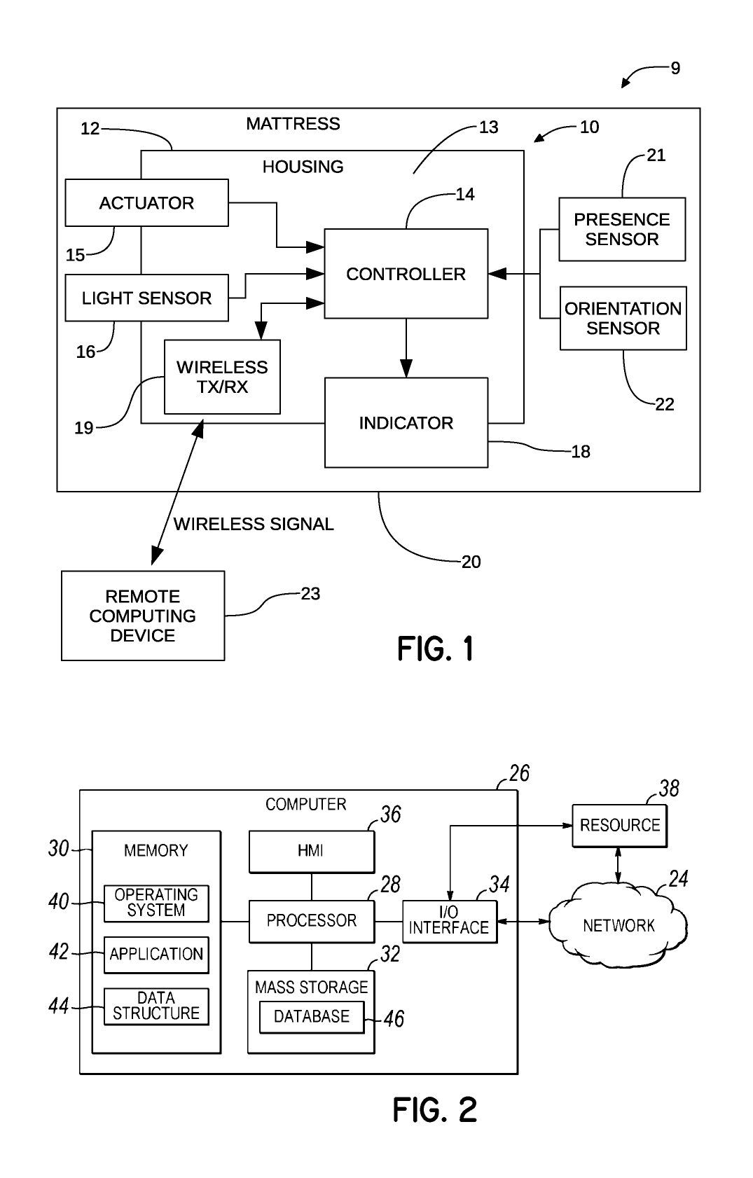

FIG. 1 illustrates an operating environment 9 that may include a tracking device 10 configured to monitor the operation of a comfort product, such as a mattress 20. Although the embodiments described herein are in the context of monitoring operation of a mattress 20, it will be appreciated that other types of comfort products may be equally applied to the described embodiments. For example and without limitation, other comfort products monitored by the tracking device 10 may include a seat cushion, a pillow, or any other like product that a user may comfortably rest on.

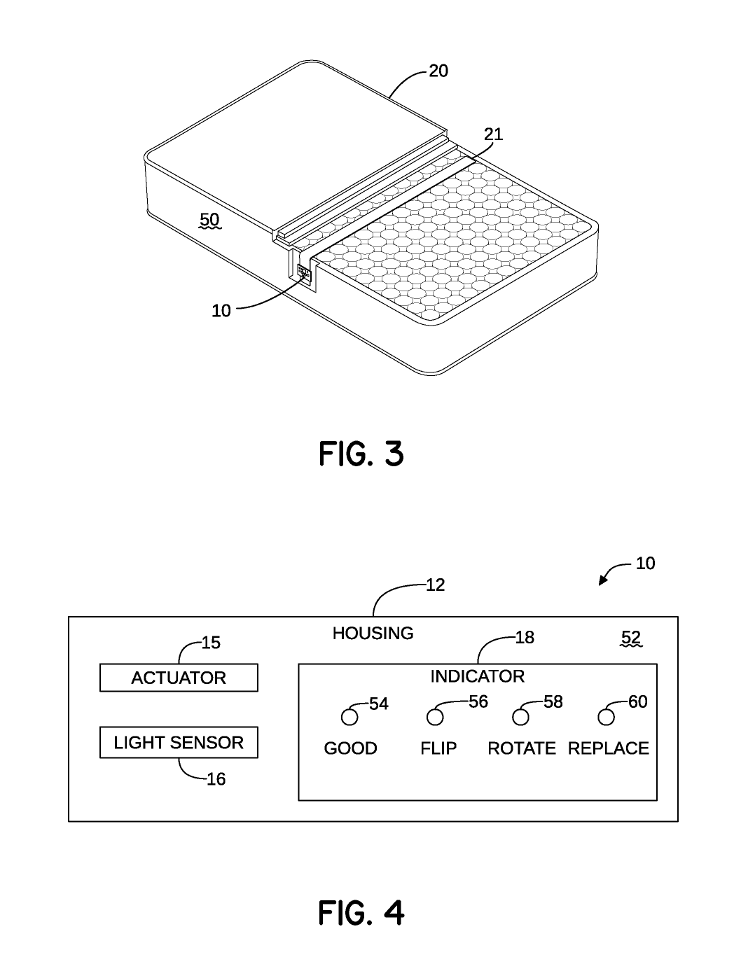

The tracking device 10 may include a housing 12 that defines an interior space 13. The housing 12 may be coupled to the mattress 20, such as by buttons, rivets, stitching, adhesive, or any other suitable method for affixing the tracking device 10 to the mattress 20. See FIG. 3. As shown in FIG. 1, the tracking device 10 may further include a controller 14, an actuator 15, a light sensor 16, and/or an indicator 18. The controller 14 may be positioned in the interior space 13 of the housing 12, and may be in electronic communication with the actuator 15, the light sensor 16, and/or the indicator 18.

The actuator 15 may include any suitable structure for receiving an input representative of an actuation. For example, the actuator 15 may include a button, a motion sensor, an audio sensor, a pressure sensor, a touch sensor, a switch, or any other suitable technology. The actuator 15 may be accessible from outside of the housing 12, meaning that input originating from outside the housing 12 may cause an actuation of the actuator 15. For example, the actuator 15 may extend outside of the housing 12 from the interior space 13, may be fully positioned in the interior space 13 of the housing 12 behind an actuation-permeable portion thereof, or may be coupled to an exterior surface of the housing 12 such that the actuator 15 is entirely positioned outside of the interior space 13. A button-type actuator 15, for example, may extend from the interior space 13 to outside the housing 12, and a user may press the button to cause an actuation. Alternatively, the button-type actuator 15 may be fully positioned in the interior space 13 of the housing 12 behind a flexible portion thereof, and a user may press the flexible portion of the housing 12 to cause an actuation. As another example, an audio sensor-type actuator 15 may be accessible from outside the housing 12 so long as the audio sensor can receive audio signals originating from outside the housing 12, such as through a portion of the housing 12 that includes a vent, an aperture, or some other audio-permeable structure.

The light sensor 16 may also be accessible from outside of the housing 12, such that the light sensor may perceive light signals originating from outside the housing 12. For example, the light sensor 16 may extend from the interior space 13 to outside the housing 12, may be fully positioned in the interior space 13 of the housing 12 behind a light-permeable portion thereof (e.g., a window), or may be coupled to an exterior surface of the housing 12 such that the light sensor 16 is entirely positioned outside of the interior space 13.

The indicator 18 may include any suitable apparatus for providing an indication relating to a mattress 20 associated with the tracking device 10. For example, the indicator 18 may include one or more LEDs, an LCD screen, a speaker, a vibration unit, or the like. The indicator 18 may be similarly accessible from outside of the housing 12 such that any indication from the indicator 18 is perceivable from outside of the housing 12. For example, if the indicator 18 is a visual indicator, then a user may view the indication from outside of the housing 12. Alternatively, if the indicator 18 is an audio indicator, then the user may hear the indication from outside of the housing 12. The indicator 18 may extend from the interior space 13 to outside of the housing 12, may be fully positioned in the interior space 13 of the housing 12 behind an indication-permeable portion thereof (e.g., a window), or may be coupled to an exterior surface of the housing 12 such that the indicator 18 is entirely positioned outside of the interior space 13.

The controller 14 may further be in electronic communication with a presence sensor 21 and/or an orientation sensor 22, each of which may be coupled to the mattress 20. The presence sensor 21 may be configured to generate a data signal indicative of whether a user or object is currently on the mattress 20. For example, the presence sensor 21 may be a capacitance sensor built into the mattress 20. The orientation sensor 22 may be configured to generate a data signal indicative of an orientation of the mattress 20. For example, the orientation sensor 22 may include an electronic compass and an electronic gyroscope. The electronic compass may be utilized to determine when the mattress 20 has been rotated, and the electronic gyroscope may be utilized to determine when the mattress 20 has been flipped.

In operation, the controller 14 may receive data from the presence sensor 21 that indicates whether a person or object is currently on the mattress 20. The controller may be configured to determine an occupancy record for the mattress 20 based on such received data. For example, the controller 14 may be configured to maintain one or more counters relating to how times the mattress 20 has been used. One counter may indicate a total number of times the mattress 20 has been used throughout the life of the mattress 20. Other counters may indicate a total number of times the mattress 20 has been used since the mattress 20 was last flipped or rotated.

Thereafter, the controller 14 may receive a check status input signal from either the actuator 15 or the light sensor 16. In one example, actuation of the actuator 15 by a user may generate the check status input signal received by the controller 14. Additionally or alternatively, detection by the light sensor 16 of a sufficient amount of light may generate the check status input signal. In response to receiving the check status input signal, the controller 14 may be configured to provide an indication of whether one or more actions relating to the mattress 20 are recommended based on the occupancy record. Possible recommended actions may include rotating the mattress 20, flipping the mattress 20, and/or replacing the mattress 20. The controller 14 may be configured to transmit a signal corresponding to the one or more recommended actions to the indicator 18, which may thereafter indicate the one or more recommended actions to the user. After the one or more recommended actions are indicated to the user, the controller 14 may communicate with the orientation sensor 22 to determine whether the one or more recommended actions have been performed.

In some embodiments, the tracking device 10 may also include a wireless transceiver/receiver 19. The wireless transceiver/receiver 19 may be positioned in the interior space 13 of the housing 12 and may be in electronic communication with the controller 14. The wireless transceiver/receiver 19 may be configured to communicate with a remote computing device 23, such as via Wi-Fi, Bluetooth, or any other suitable protocol. In this way, the controller 14 is enabled to wirelessly transmit information relating to the mattress 20, such as presence data, orientation data, an occupancy record, whether an action relating to the mattress 20 is recommended, or whether a recommended action has been performed, to the remote computing device 23. As some non-limiting examples, the remote computing device 23 may include a laptop computer, a desktop, a tablet, a cellular phone, or any like device suitable for receiving information from the tracking device 10.

Upon or after receiving information from the tracking device 10, the remote computing device 23 may be configured to provide an indication relating to the mattress 20, such as an indication of whether rotating, flipping, or replacing the mattress 20 is recommended. In some embodiments, information is automatically transmitted between the tracking device 10 and the remote computing device 23 on an event-driven basis, such as when a presence is detected on the mattress 20 or when there is a recommended action for the mattress 20. In addition or alternatively, information may be transmitted at regular intervals or on-demand from the remote computing device 23.

The remote computing device 23 may operate in conjunction with the actuator 15 and the indicator 18 of the tracking device 10. Alternatively, the remote computing device 23 may replace one or more of these elements. In other words, the remote computing device 23 may function as the actuator 15 and/or the indicator 18. For example, the remote computing device 23 may include an actuator 15 implemented in hardware and/or software. Upon actuation of the actuator 15 on the remote computing device 23, the remote computing device 23 may be configured to transmit a signal indicative of the actuation to the controller 14 of the tracking device 10 via the wireless transceiver/receiver 19. Furthermore, the remote computing device 23 may be configured to generate an indication via an indicator of the remote computing device 23, such as a display screen, relating to the mattress 20. For example, the remote computing device 23 may indicate that the mattress 20 is recommended to be flipped, rotated, or replaced.

The tracking device 10 and/or the remote computing device 23 may offer an improved mechanism for determining when a mattress 20 should be flipped, rotated, or replaced. In particular, the useful lifespan of a mattress may largely depend on the operation (or use) of the mattress 20. For example, a good quality mattress 20 that receives proper care may last about ten years of nightly use. However, if the mattress 20 is used only periodically, then the useful lifespan of the mattress 20 may expand well beyond ten years. Thus, by tracking operation of the mattress 20 rather than the mere passage of time, the tracking device 10 may offer a more precise mechanism for determining when the mattress 20 should be flipped, rotated, or replaced. Tracking a mattress 20 by the operation thereof may be especially useful in a contract bedding scenario, such as in a hotel, where a given mattress 20 may be used on an irregular basis depending on the hotel's occupancy.

Referring to FIG. 2, the controller 14 and/or remote computing device 23 may be implemented on one or more computer devices or systems, such as exemplary computer system 26. The computer system 26 may include a processor 28, a memory 30, a mass storage memory device 32, an input/output (I/O) interface 34, and a Human Machine Interface (HMI) 36. The computer system 26 may also be operatively coupled to one or more external resources 38 via the network 24 or I/O interface 34. External resources may include, but are not limited to, servers, databases, mass storage devices, peripheral devices, cloud-based network services, or any other suitable computer resource that may be used by the computer system 26. In one example, the I/O interface 34 may include the wireless transceiver/receiver 19, and the external resource 38 may include the remote computing device 23.

The processor 28 may include one or more devices selected from microprocessors, micro-controllers, digital signal processors, microcomputers, central processing units, field programmable gate arrays, programmable logic devices, state machines, logic circuits, analog circuits, digital circuits, or any other devices that manipulate signals (analog or digital) based on operational instructions that are stored in the memory 30. Memory 30 may include a single memory device or a plurality of memory devices including, but not limited to, read-only memory (ROM), random access memory (RAM), volatile memory, non-volatile memory, static random access memory (SRAM), dynamic random access memory (DRAM), flash memory, cache memory, or any other device capable of storing information. The mass storage memory device 32 may include data storage devices such as a hard drive, optical drive, tape drive, non-volatile solid state device, or any other device capable of storing information.

Processor 28 may operate under the control of an operating system 40 that resides in memory 30. The operating system 40 may manage computer resources so that computer program code embodied as one or more computer software applications, such as an application 42 residing in memory 30, may have instructions executed by the processor 28. In an alternative embodiment, the processor 28 may execute the application 42 directly, in which case the operating system 40 may be omitted. One or more data structures 44 may also reside in memory 30, and may be used by the processor 28, operating system 40, or application 42 to store or manipulate data.

The I/O interface 34 may provide a machine interface that operatively couples the processor 28 to other devices and systems, such as the network 24 or external resource 38. The application 42 may thereby work cooperatively with the network 24 or external resource 38 by communicating via the I/O interface 34 to provide the various features, functions, applications, processes, or modules comprising embodiments of the invention. The application 42 may also have program code that is executed by one or more external resources 38, or otherwise rely on functions or signals provided by other system or network components external to the computer system 26. Indeed, given the nearly endless hardware and software configurations possible, persons having ordinary skill in the art will understand that embodiments of the invention may include applications that are located externally to the computer system 26, distributed among multiple computers or other external resources 38, or provided by computing resources (hardware and software) that are provided as a service over the network 24, such as a cloud computing service.

The HMI 36 may be operatively coupled to the processor 28 of computer 26 in a known manner to allow a user to interact directly with the computer 26. The HMI 36 may include video or alphanumeric displays, a touch screen, a speaker, and any other suitable audio and visual indicators capable of providing data to the user. The HMI 36 may also include input devices and controls such as an alphanumeric keyboard, a pointing device, keypads, pushbuttons, control knobs, microphones, etc., capable of accepting commands or input from the user and transmitting the entered input to the processor 28. In one example, the HMI 36 may include the actuator 15, the light sensor 16, and/or the indicator 18.

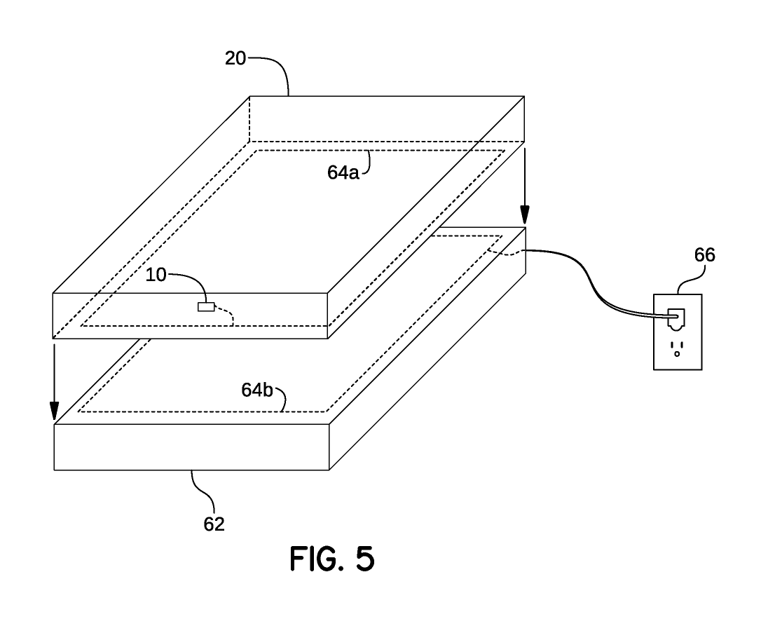

A database 46 may reside on the mass storage memory device 32, and may be used to collect and organize data used by the various systems and modules described herein. The database 46 may include data and supporting data structures that store and organize the data. In particular, the database 46 may be arranged with any database organization or structure including, but not limited to, a relational database, a hierarchical database, a network database, or combinations thereof. A database management system in the form of a computer software application executing as instructions on the processor 28 may be used to access the information or data stored in records of the database 46 in response to a query, where a query may be dynamically determined and executed by the operating system 40, other applications 42, or one or more modules. In one example, the database 46 may comprise a database of one or more threshold values in which to compare one or more sleep cycle counters 74 (FIG. 5) to determine whether the mattress 20 should be rotated, flipped, or replaced.

Referring to FIG. 3, the tracking device 10 may be coupled to the mattress 20 on a side surface 50 thereof. In this way, a user may lie on the mattress 20 without receiving potential discomfort caused by making contact with the tracking device 10. Alternatively, the tracking device 10 may be coupled to other areas of the mattress 20, such as on a top surface in a corner. In the illustrated embodiment, the presence sensor 21 runs through the center of the mattress 20 and is coupled to the tracking device 10. Additional presence sensors 21 may run through other areas of the mattress 20 to maximize the detection of persons or objects thereon.

FIG. 4 illustrates a surface 52 of the tracking device 10 that may permit access to the actuator 15, the light sensor 16, and/or the indicator 18. The indicator 18 may be a visual indicator that includes at least one Light Emitting Diode (“LED”). As previously described, the indicator 18 may provide an indication to a user of whether an action relating to the mattress 20 is recommended. Such indication may be provided in response to the controller 14 receiving a check status input signal, such as from the actuator 15 after being sufficiently actuated or the light sensor 16 after detecting a sufficient amount of light. In one example, the indicator 18 may include LEDs 56 and 58. Each of LEDs 56 and 58 may be capable of illuminating in multiple colors, such as red and green. LED 56 may be labeled “FLIP,” and LED 58 may be labeled “ROTATE.” A green illumination of either of the LEDs 56 and 58 may indicate that the action associated with that LED, such as FLIP or ROTATE, is not yet recommended. Conversely, a red illumination of one of the LEDs 56 and 58 may indicate that the action associated with the LED is recommended. A red illumination of both LEDs 56 and 58 may indicate that replacement of the mattress 20 is recommended.

In an alternative example, the indicator 18 may include additional LEDs 54 and 60. The LED 54 may be labeled “GOOD,” and the LED 60 may be labeled “REPLACE.” In this alternative example, each of the LEDs 54, 56, 58, and 60 may be capable of illuminating in a single color, such as green or red. For example, the GOOD LED 54 may be capable of illuminating green, and the other LEDs 56, 58, and 60 may be capable of illuminating red. Upon the controller 14 receiving the check status input signal, the LED 54 labeled GOOD may illuminate green if no actions relating to the mattress 20 are recommended. Alternatively, if flipping, rotating, or replacing the mattress 20 is recommended, the LEDs 56, 58, or 60 may illuminate red respectively. Illumination of any of the LEDs 54, 56, 58, and 60, as described above, may be in a solid or blinking manner. For example, when illuminated, any one of the LEDs 54, 56, 58, and 60 may continue to emit light for a set period of time, or instead blink on and off for a set period of time.

Referring to FIG. 5, the mattress 20 may be adapted to be placed upon a foundation 62, which may be a fixed or semi-permanent base. For example, the foundation 62 may be a bedspring, a platform, a bed frame, etc. The foundation 62 may have a top surface that is of a similar size and shape to a bottom surface of the mattress 20 so as to enable the mattress 20 to be aligned with and rest upon the foundation 62. In this way, the foundation 62 may support the mattress 20 when in use.

As previously described, the mattress 20 may include one or more electronic components, such as the tracking device 10, the presence sensor 21, and the orientation sensor 22. In one embodiment, the mattress 20 may include one or more power sources, such as a battery, for powering the mattress 20 and/or any electronic components included therewith. However, some users may feel uncomfortable utilizing a mattress 20 that includes a built-in power source. Accordingly, in another embodiment, the foundation 62 may be configured to supply power to the mattress 20 from a power source, such as a power outlet 66, that is external to the mattress 20. More particularly, the foundation 62 may be coupled to or include a power source external to the mattress 20, and may supply power from the power source to the mattress 20 when the mattress 20 is positioned upon the foundation 62. The power supplied to the mattress 20 from the foundation 62 may then be utilized to power the mattress 20 and/or any electronic components included therewith. In alternative embodiments, the mattress 20 may be directly coupled to an external power source, such as the power outlet 66, and/or may include built-in power sources that supply power in conjunction with an external power source and/or the foundation 62.

The foundation 62 may be configured to supply power to the mattress 20 via an inductive coupling formed therebetween. In particular, the mattress 20 may include a receiving coil 64a that is embedded therein adjacent to a bottom surface thereof, and the foundation 62 may include a transmitter coil 64b embedded therein adjacent to a top surface thereof. The mattress 20 and the foundation 62 may further include any other electrical components generally known to facilitate the creation of an inductive coupling between two devices. The mattress 20 and the foundation 62 may be configured such that, when the mattress 20 is aligned with and placed upon the foundation 62, the receiving coil 62a and the transmitter coil 64b are approximated with one another. When such approximation occurs, the transmitter coil 64b may generate an electromagnetic field that enables energy, such as from the power outlet 66, to be transmitted from the transmitter coil 64b to the receiving coil 64a. The energy received at the receiving coil 64a may then be supplied to electronic components of the mattress 20 that are electrically coupled to the receiving coil 64a.

The inductive coupling formed between the mattress 20 and the foundation 62 is configured such that energy may be transmitted from the transmitter coil 64b to the receiving coil 64a through non-metal materials, such as fabric. Accordingly, the receiving coil 64a and the transmitter coil 64b need not be in physical contact with one another, but only approximated with another, for energy to be transmitted therebetween. This ability to transfer energy without physical contact enables the receiving coil 64a and the transmitter coil 64b to be fully embedded within the mattress 20 and the foundation 62, respectively, and enables the mattress 20 to be hermetically sealed for an increased lifespan. This ability also eliminates the need for cables running between the mattress 20 and the foundation 62, and thereby enables the mattress 20 to be moved or rotated relative to the foundation 62 without first unplugging or disconnecting cables. Moreover, because the receiving coil 64a and the transmitter coil 64b need only be approximated with one another, loose or imperfect alignment between the mattress 20 and the foundation 62 is sufficient to enable formation of the inductive coupling. Thus, even if the mattress 20 moves slightly out of alignment with the foundation 62, energy may still be transmitted therebetween.

The mattress 20 and the foundation 62 may be configured such that, when the mattress 20 is at least loosely aligned and placed upon the foundation 62, the receiving coil 64a and the transmitter coil 64b are approximated with one another notwithstanding the specific rotation of the mattress 20 relative to the foundation 62. In the illustrated embodiment, for example, the receiving coil 64a runs along the perimeter of the lower surface of the mattress 20, and the transmitter coil 64b runs along the perimeter of the top surface of the foundation 62. Hence, even if the mattress 20 is rotated relative to the foundation 62 by one hundred eighty degrees, which helps distribute wear of the mattress 20 and thereby prolong the life thereof, the receiving coil 64a and the transmitter coil 64b will continue to be approximated with respect to one another after the rotation is complete. It will be appreciated that other configurations of the receiving coil 64a and the transmitter coil 64b are equally effective for this purpose. For example, in alternative embodiments, one or more receiving coils 64a and one or more transmitter coils 64b may be respectively positioned in the center of the mattress 20 and the foundation 62, in two or more corners of each the mattress 20 and the foundation 62, or adjacent to two or more sides of each of the mattress 20 and the foundation 62.

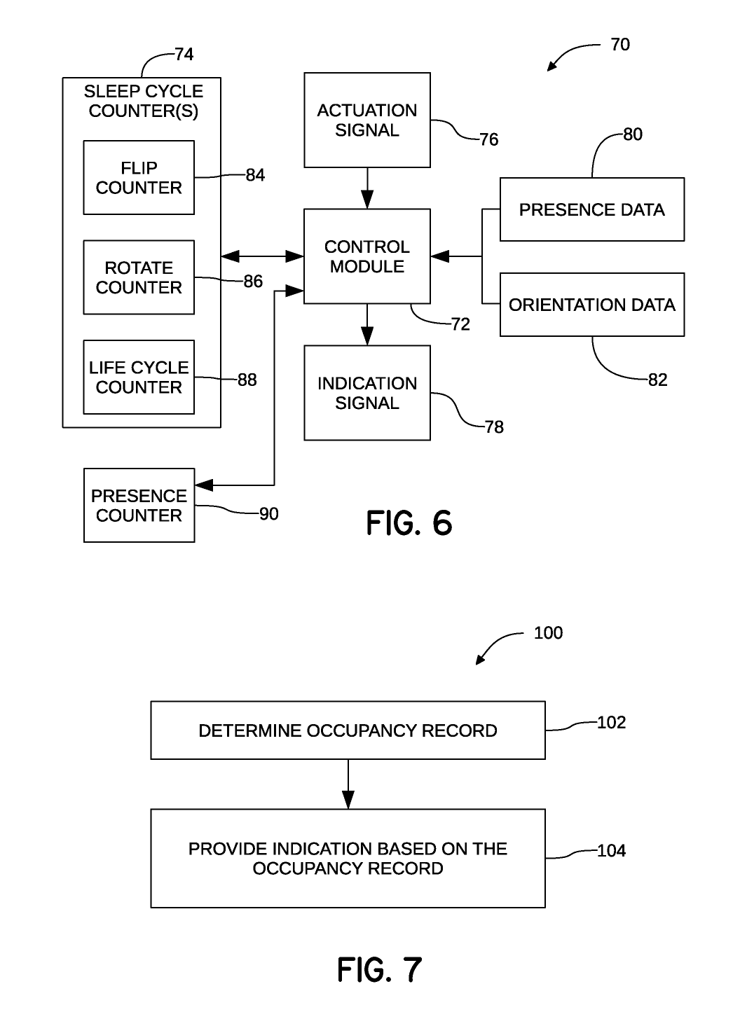

FIG. 6 illustrates a mattress tracking system 70 that may include a control module 72, one or more sleep cycle counters 74, and/or a presence counter 90. The mattress tracking system 70 may be provided by the tracking device 10 and/or the remote computing device 23 of the operating environment 9.

The control module 72 may receive an actuation signal 76, such as from the actuator 15 or the light sensor 16. In response to receiving the actuation signal 76, the control module 72 may take various actions based on the type of actuation signal 76 that is received. For example, if the actuation signal 76 represents an initiation input, the control module 72 may initialize the one or more sleep cycle counters 74 and the presence counter 90 to zero. Similarly, if the actuation signal 76 represents a reset input signal, the control module 72 may reset the one or more sleep cycle counters 74 and the presence counter 90 to zero. And if the actuation signal 76 represents a status check input, the control module 72 may compare the one or more sleep cycle counters 74 to one or more preprogrammed thresholds. The control module 72 may generate an indication signal 78 that represents one or more recommended actions relating to a mattress 20 based on the comparison(s). This indication signal 78 may be transmitted to the indicator 18, which may then provide an indication of the one or more recommended actions to a user.

The one or more sleep cycle counters 74 may include a flip counter 84, a rotate counter 86, and/or a life cycle counter 88. Each of these counters may be utilized in tracking the use of a mattress 20 and determining whether an action relating to the mattress 20 is recommended. For example, the control module 72 may receive presence data 80, such as from the presence sensor 21, that indicates whether a person or object is currently on the mattress 20. In response to receiving presence data 80 that indicates a person or object is currently on the mattress 20, the control module 72 may be configured to increment the one or more sleep cycle counters 74.

After determining and indicating that an action relating to the mattress 20 is recommended, the control module 72 may receive orientation data 82, such as from the orientation sensor 22. The control module 72 may utilize the orientation data 82 to determine whether the recommended action has been performed. If so, the control module 72 may note that the recommended action has been performed by taking appropriate action, such as by adjusting the values of one or more sleep cycle counters 74.

FIG. 7 illustrates a flowchart of a process 100 for tracking the operation of a mattress 20 that may be performed by the mattress tracking system 70. In block 102, an occupancy record may be determined, such as by the control module 72, for the mattress 20. In particular, the control module 72 may determine the occupancy record based on received presence data 80. As previously described, the received presence data 80 may indicate when a person or object is on the mattress 20. In block 104, an indication may be provided of whether one or more actions relating to the mattress 20 are recommended based on the occupancy record. In particular, the control module 72 may generate an indication signal 78 representative of the one or more recommended actions. The indication signal 78 may be transmitted to the indicator 18, which may then indicate the one or more recommended actions to the user.

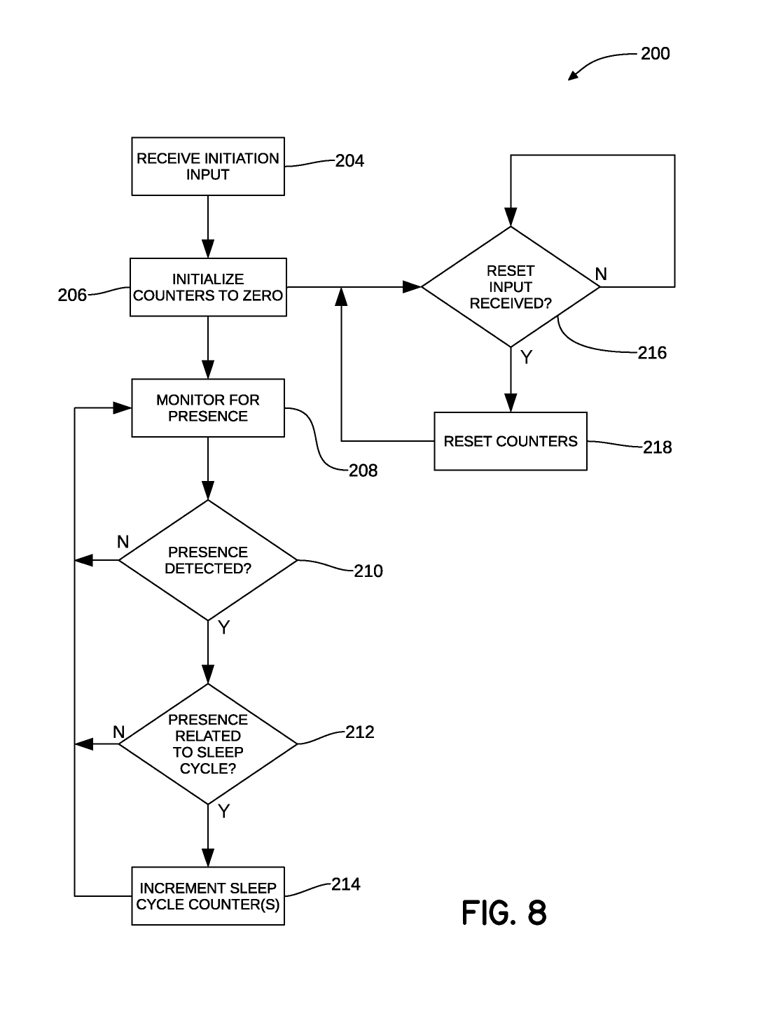

FIG. 8 illustrates a flowchart of a process 200 for determining the occupancy record in block 102 of the process 100. In block 204, an initiation input signal may be received, such as at the control module 72. The initiation input signal may be generated by an actuation of the actuator 15 for a set period of time, such as ten seconds. Alternatively, the initiation input signal may be generated by the light sensor 16 in response to the light sensor 16 being exposed to a sufficient amount of light. For example, a new mattress 20 may include a tracking device 10 with a cover (e.g., tape) applied over the light sensor 16. Upon receiving the new mattress 20, and before casing the mattress with sheets, a user may remove the cover from the light sensor 16 to expose the light sensor 16 to light. Upon being exposed to a sufficient amount of light for a set period of time, such as ten seconds, the light sensor 16 may generate the initiation input signal.

In block 206, in response to receiving the initiation input signal, the one or more sleep cycle counters 74 and the presence counter 90 may be initialized to zero. Thereafter, in block 208, monitoring for a presence may be performed. In one example, the control module 72 may be configured to monitor for the presence by querying the presence sensor 21 for presence data 80 at set timer intervals, such as every hour. By querying the presence sensor 21 for presence data 80 at set time intervals, as opposed to continuously querying the presence sensor 21, the mattress tracking system 70 is able to operate while consuming less power.

The presence data 80 received from the presence sensor 21 may indicate whether or not a presence, such as a person or object, is currently detected on a mattress 20. Thus, in block 210, a determination may be made from the presence data 80 as to whether a presence is detected on the mattress 20. If not, then monitoring for a presence may be continued at block 208. If a presence is detected on the mattress 20, then at block 212, a determination may be made as to whether the presence relates to a sleep cycle. Determining whether a presence relates to a sleep cycle may include determining if a presence is detected on the mattress 20 for a set period time, which in turn may include determining if a presence is detected on the mattress 20 at the end of each of a set number of consecutive time intervals following an initial presence detection. If the presence is detected for the set period of time, the control module 72 may determine that the presence relates to a sleep cycle. In response to determining that the presence relates to a sleep cycle, the one or more sleep cycle counters 74 may be incremented at block 214. If not, then monitoring for a presence may be continued at block 208.

After the one or more sleep cycle counters 74 and the presence counter 90 have been initialized to zero at block 206, monitoring for receipt of a reset input signal may be performed, such as by the control module 72, at block 216. The reset input signal may be generated by an actuation of the actuator 15 for a set period of time, or alternatively may be generated by the light sensor 16 being exposed to sufficient light for a set period of time. The set period of time for generating the reset input signal may be equal to the set period of time for generating the initiation input signal. If the reset input signal is received, the one or more sleep cycle counters 74 and the presence counter 90 may be reset to zero at block 218. In this way, a user, if desired, may reset the tracking device 10 and thereby cancel any currently recommended actions. Upon receipt of either the reset input signal or the initiation input signal, sensors in the mattress, such as the presence sensor 21 or the orientation sensor 22, may automatically be calibrated, and the indicator 18 may provide an indication of such receipt. For example, in the case of a visual indicator 18 having two or more LEDs, the LEDs may perform a dance in which the LEDs blink on and off in a predetermine sequence.

Returning to FIG. 7, the occupancy record determined in block 102 of the process 100 may include the one or more sleep cycle counters 74, which may generally track a number of times a mattress 20 has been used relative to sleep cycles. In one example, the occupancy record may include a single sleep cycle counter 74 that tracks overall use of the mattress 20 relative to sleep cycles. Alternatively, the occupancy record may include multiple sleep cycle counters 74, such as the flip counter 84, the rotate counter 86, and/or the life cycle counter 88. Either way, providing the indication of whether an action relating to the mattress is recommended in block 104 of the process 100 may be based on the one or more sleep cycle counters 74 of the occupancy record determined in block 102.

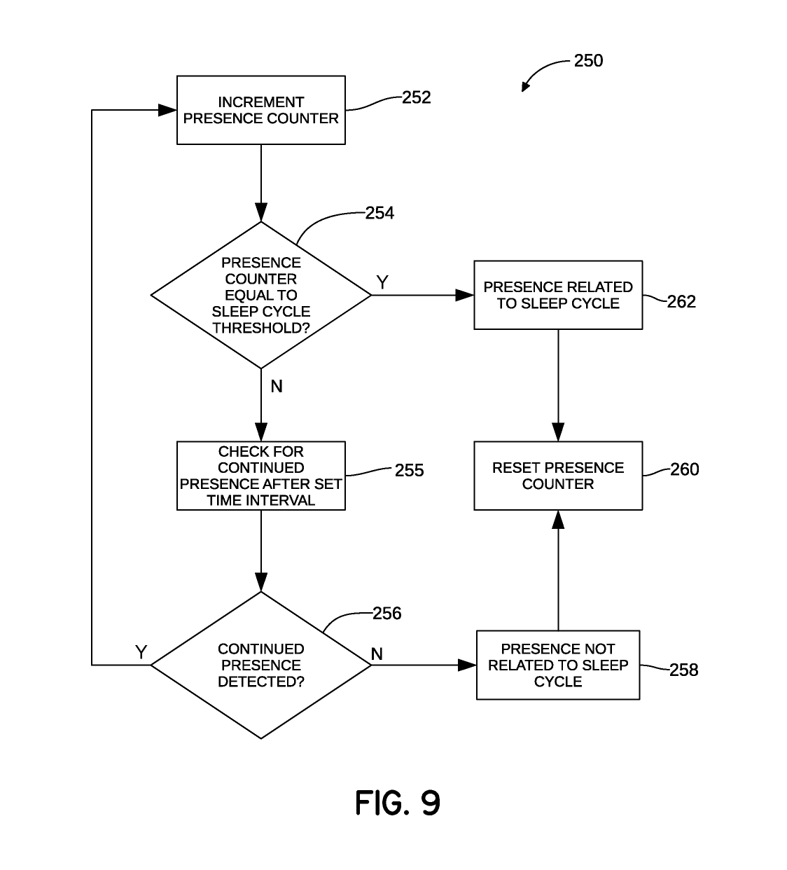

FIG. 9 illustrates a flowchart of a process 250 for determining whether a detected presence relates to a sleep cycle in block 212 of the process 200. At block 252, upon an initial detection of a presence on the mattress 20, the presence counter 90 may be incremented or increased. At block 254, a determination may be made as to whether the presence counter 90 has reached a sleep cycle threshold value. In response to determining that the presence counter 90 has not yet reached the sleep cycle threshold value, at block 255, it may be checked whether a presence, which may be a presence that is the same as or different from the last detected presence, continues to be detected on the mattress 20 at the end of a set time interval following the previous presence detection. More particularly, the control module 72 may be configured to query the presence sensor 21 for presence data 80 at the end of the set time interval following the previous presence detection. For example, the control module 72 may query the presence sensor 21 for presence data 80 an hour following the initial presence detection.

At block 256, a determination may be made, such as based on the presence data 80, whether a presence continues to be detected on the mattress 20 at the end of the set time interval following the previous presence detection. In response to a continued presence being detected at the end of the set time interval, the presence counter 90 may again be incremented at block 252. Thereafter, at block 254, a determination may again be made as to whether the presence counter 90 is equal to the sleep cycle threshold value. Alternatively, in response to a continued presence not being detected at the end of the set time interval at block 256, a determination may be made that the presence does not relate to a sleep cycle at block 258. Thereafter, at block 260, the presence counter 90 may be reset to zero.

In response to determining that the presence counter 90 has reached the sleep cycle threshold value at block 254, a determination may be made that the presence relates to a sleep cycle at block 262. Thereafter, at block 260, the presence counter 90 may be reset to zero.

The process 250 enables the control module 72 to base the occupancy record on detections of consecutive presences on the mattress 20 rather than detections of minimal or non-consecutive presences. In this way, the control module 72 may not increment the one or more sleep cycle counters 74 based on presences on the mattress 20 that are minimal or non-consecutive, as such minimal or non-consecutive presences may not cause patterns of wear in the mattress 20 to the same extent as ongoing or consecutive presences. For example, if the sleep cycle threshold value is set to four, and the presence check is performed every hour, then the control module 72 may increment the one or more sleep cycle counters 74 in response to a presence being detected on the mattress 20 at the start of and after each of three consecutive hours. Conversely, in some embodiments, the control module 72 may increment the one or more sleep cycle counters 74 in response to any detected presence on the mattress 20, whether consecutive or not. This may occur if block 212, namely determining whether the presence is related to a sleep cycle, is omitted from the process 200, or if the sleep cycle threshold value is set to one.

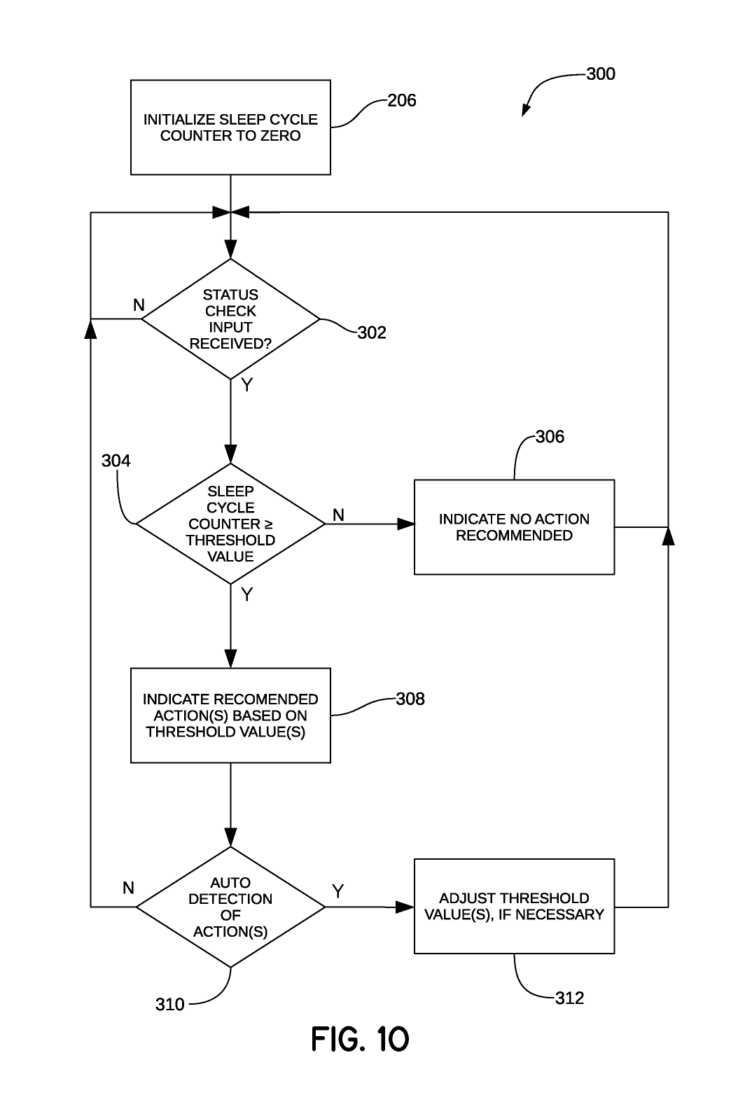

FIG. 10 illustrates a flowchart of a process 300 for providing an indication of whether an action relating to the mattress 20 is recommended based on the occupancy record in block 104 of the process 100. In the process 300, a single sleep cycle counter 74 may be utilized to determine whether an action relating to the mattress 20 is recommended. After the sleep cycle counter 74 and the presence counter 90 is initialized to zero at block 206, as described for the process 200, a determination may be made as to whether a status check input signal is received at block 302. The status check input signal may be generated by an actuation of the actuator 15, such as for a set period of time. Alternatively, the status check input signal may be generated by the light sensor 16 in response to the light sensor 16 being exposed to sufficient light, such as for a set period of time. In this way, when a user removes sheets from the mattress 20, the check status input signal may be automatically generated by the light sensor 16.

In some embodiments, the check status input signal may only be generated when the light sensor 16 is exposed to sufficient light, such as for a set period of time. This ensures that the check status input signal is not generated in response to an accidental actuation of the actuator 15, such as when the indicator 18 is covered by sheets or when a user is sleeping. The set period of time for generating the check status input signal may be less than the set period of time for generating the initiation input signal and the reset input signal. For example, the set period of time for generating the check status input signal may be two milliseconds.

In response to receiving the check status input signal, a determination may be made as to whether the sleep cycle counter 74 is greater than or equal to one or more threshold values at block 304. Such threshold values may be preprogrammed in advance. For example, one threshold value may correspond to a recommendation to rotate the mattress 20, one threshold value may correspond to a recommendation to flip the mattress 20, and one threshold value may correspond to a recommendation to replace the mattress 20. If the sleep cycle counter 74 is not greater than or equal to any of the threshold values, an indication that no action is recommended may be provided, such as via the indicator 18, at block 306.

Alternatively, at block 308, if the sleep cycle counter 74 is greater than or equal to one or more of the threshold values, a recommended action may be indicated, such as via the indicator 18, based on which threshold values the sleep cycle counter 74 equals or exceeds. For example, if the sleep cycle counter 74 exceeds the threshold value associated with flipping the mattress 20, the control module 72 may transmit an indication signal 78 representative of a recommendation to flip the mattress 20 to the indicator 18. The indicator 18 may then provide an indication of the recommended action according to the received indication signal 78.

In block 310, it may be automatically determined whether a recommended action, such as a recommendation to flip or rotate the mattress 20, has been performed. For example, the control module 72 may receive orientation data 82 from the orientation sensor 22 to determine whether the mattress 20 has been rotated or flipped. If so, then at block 312, one or more of the threshold values may be adjusted. For example, if a recommendation to flip the mattress 20 has been satisfied, then the threshold value corresponding to the recommendation to flip the mattress 20 may be increased to a value for the sleep cycle counter 74 to reach before the mattress 20 should be flipped again.

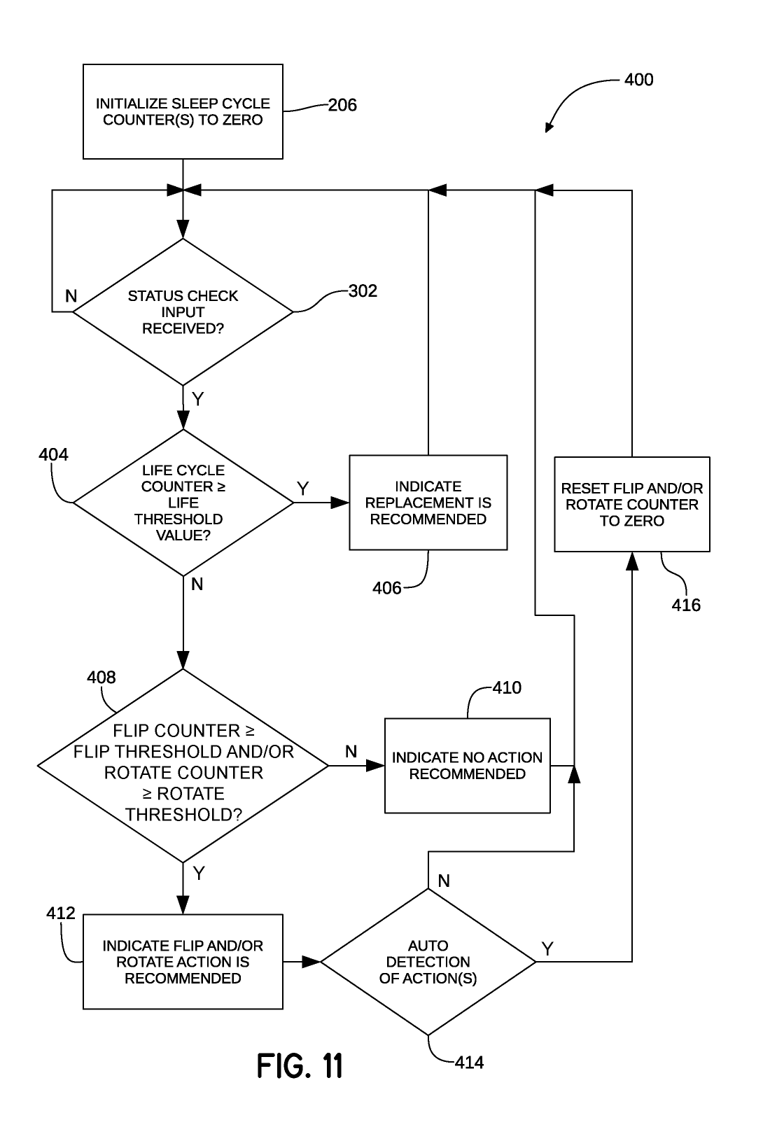

FIG. 11 illustrates a flowchart of another process 400 for providing an indication of whether an action relating to the mattress 20 is recommended based on the occupancy record in block 104 of the process 100. In the process 400, multiple sleep cycle counters 74 may be utilized to determine whether an action relating to a mattress 20 is recommended. The multiple sleep cycle counters 74 may include the flip counter 84, the rotate counter 86, and the life cycle counter 88.

After the sleep cycle counters 74 and the presence counter 90 have been initialized to zero in block 206, as described for the process 200, and the check status input signal has been received at block 302, as described for the process 300, a determination may be made of whether the life cycle counter 88 is greater than or equal to a life cycle threshold value at block 404. In response to a determination that the life cycle counter 88 equals or exceeds the life cycle threshold value, at block 406, an indication that replacement of the mattress 20 is recommended may be provided to the user, such as via the indicator 18. Alternatively, if the life cycle counter 88 is not determined to be greater than or equal to the life cycle threshold, then a determination may be made of whether the flip counter 84 and/or the rotate counter 86 is greater than or equal to a flip threshold value and/or a rotate threshold value, respectively, at block 408.

If neither the flip counter 84 nor the rotate counter 86 is greater than or equal to their respective threshold values, then an indication may be provided, such as via the indicator 18, that no action is recommended relative to the mattress 20 at block 410. Alternatively, in response to the flip counter 84 and/or the rotate counter 86 reaching or exceeding their respective threshold values, then an indication to flip and/or rotate the mattress 20 may be respectively provided, such as via the indicator 18, at block 412. Thereafter, at block 414, a determination may be automatically made as to whether a recommended action, such as a recommendation to flip and/or rotate the mattress 20, has been performed. Block 414 may be equivalent to block 310 of the process 300. In response to automatically determining that the mattress 20 has been flipped and/or rotated, then at block 416, the sleep cycle counter 74 corresponding to the detected action (e.g., the flip counter 84 and/or the rotate counter 86), may be reset to zero at block 416.

In an alternative embodiment, the sleep cycle counters 74 may include two counters: a life cycle counter 88 and a combined counter for determining whether flipping or rotating of the mattress 20 is recommended. In this embodiment, the process for providing an indication of whether an action relating to the mattress 20 is recommended based on the occupancy record is similar to the process 400 except that, to determine whether it is recommended to flip or rotate the mattress 20, the combined counter may be compared with a set threshold value. Whenever the combined counter equals or exceeds the set threshold value, an indication may be provided, such as via the indicator 18, that alternates between providing a recommendation to flip the mattress 20 and providing a recommendation to rotate the mattress 20. Hence, in one example, when the combined counter first reaches the set threshold value, the control module 72 may generate an indication signal 78 that corresponds to a recommendation to rotate the mattress 20. Thereafter, upon rotation of the mattress 20 being automatically detected, the combined counter may be reset to zero. Then, upon the combined counter again reaching the set threshold value, the control module 72 may generate an indication signal 78 that now corresponds to a recommendation to flip the mattress 20. Thereafter, upon flipping of the mattress 20 being automatically detected, the combined counter may again be reset to zero. This cycle, or vice versa, may continue until the life cycle counter 88 reaches a set threshold value, which indicates that replacement of the mattress 20 is recommended.

The processes 300 and 400 described above each determine whether an action relating to the mattress 20 is recommended in response to receiving a status check input signal. In alternative embodiments, however, this determination may be made automatically in response to any of the one or more sleep cycle counters 74 being increased. For example, whenever a presence is determined to be related to a sleep cycle (block 212 of the process 200), and consequently the one or more sleep cycle counters 74 are incremented (block 214 of the process 200), the one or more sleep cycle counters 74 may automatically be compared with the appropriate thresholds, as described above. If the comparison(s) indicate that one or more actions are recommended for the mattress 20, the control module 72 may set one or more flags that correspond to the one or more recommended actions. Thereafter, when the check status input signal is received, the control module 72 may read the set flags and generate an indication signal 78 representative of the recommended actions corresponding to the set flags. The control module 72 may provide this indication signal 78 to the indicator 18, which may in turn provide the one or more recommended actions to the user. By determining whether an action relating to the mattress 20 is recommended before the check status input signal is received, processing time upon receiving the check status input signal is reduced. Similar to block 312 of the process 300 and block 414 of the process 400, performance of a recommended action may be automatically detected. In response to a recommended action being automatically detected, the flag and/or the sleep cycle counter 74 corresponding to the detected action may be reset.

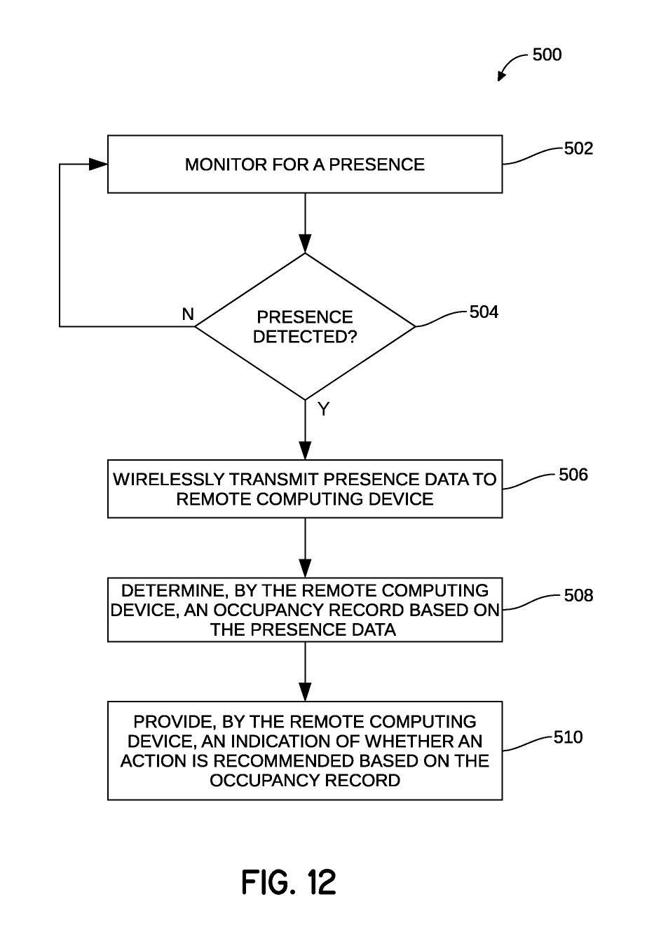

FIG. 12 illustrates a flowchart of a process 500 for tracking the operation of the mattress 20 that uses a remote computing device 23. The process 500 may be performed by the mattress tracking system 70. At block 502, monitoring for a presence on the mattress 20 may be performed. For example, the control module 72 may query the presence sensor 21 for presence data 80 that indicates whether a presence is currently on the mattress. At block 504, a determination may be made as to whether a presence is detected on the mattress 20, such as from the presence data 80. If a presence is not detected, then at block 502, continued monitoring for a presence may be performed. For example, after a set time interval, such as an hour, the control module 72 may again query the presence sensor 21 for presence data 80.

In response to a determination that a presence is detected, the data indicating the presence, such as the presence data 80, may be wireless transmitted to the remote computing device 23, such as via the wireless transceiver/receiver 19, at block 506. Thereafter, at block 508, the remote computing device 23 may determine an occupancy record for the mattress 20 based on the received presence data. For example, the remote computing device 23 may implement one or more of the blocks of the process 200 for this purpose. At block 510, the remote computing device 23 may provide an indication of whether an action is recommended for the mattress 20 based on the occupancy record. For example, the remote computing device 23 may implement one or more of the blocks of the processes 300 or 400 for this purpose.

In general, the routines executed to implement the embodiments of the invention, whether implemented as part of an operating system or a specific application, component, program, object, module or sequence of instructions, or even a subset thereof, may be referred to herein as “computer program code,” or simply “program code.” Program code typically comprises computer readable instructions that are resident at various times in various memory and storage devices in a computer and that, when read and executed by one or more processors in a computer, cause that computer to perform the operations necessary to execute operations and/or elements embodying the various aspects of the embodiments of the invention. Computer readable program instructions for carrying out operations of the embodiments of the invention may be, for example, assembly language or either source code or object code written in any combination of one or more programming languages.

Various program code described herein may be identified based upon the application within that it is implemented in specific embodiments of the invention. However, it should be appreciated that any particular program nomenclature that follows is used merely for convenience, and thus the invention should not be limited to use solely in any specific application identified and/or implied by such nomenclature. Furthermore, given the generally endless number of manners in which computer programs may be organized into routines, procedures, methods, modules, objects, and the like, as well as the various manners in which program functionality may be allocated among various software layers that are resident within a typical computer (e.g., operating systems, libraries, API's, applications, applets, etc.), it should be appreciated that the embodiments of the invention are not limited to the specific organization and allocation of program functionality described herein.

The program code embodied in any of the applications/modules described herein is capable of being individually or collectively distributed as a program product in a variety of different forms. In particular, the program code may be distributed using a computer readable storage medium having computer readable program instructions thereon for causing a processor to carry out aspects of the embodiments of the invention.

Computer readable storage media, which is inherently non-transitory, may include volatile and non-volatile, and removable and non-removable tangible media implemented in any method or technology for storage of information, such as computer-readable instructions, data structures, program modules, or other data. Computer readable storage media may further include RAM, ROM, erasable programmable read-only memory (EPROM), electrically erasable programmable read-only memory (EEPROM), flash memory or other solid state memory technology, portable compact disc read-only memory (CD-ROM), or other optical storage, magnetic cassettes, magnetic tape, magnetic disk storage or other magnetic storage devices, or any other medium that can be used to store the desired information and which can be read by a computer. A computer readable storage medium should not be construed as transitory signals per se (e.g., radio waves or other propagating electromagnetic waves, electromagnetic waves propagating through a transmission media such as a waveguide, or electrical signals transmitted through a wire). Computer readable program instructions may be downloaded to a computer, another type of programmable data processing apparatus, or another device from a computer readable storage medium or to an external computer or external storage device via a network.

Computer readable program instructions stored in a computer readable medium may be used to direct a computer, other types of programmable data processing apparatus, or other devices to function in a particular manner, such that the instructions stored in the computer readable medium produce an article of manufacture including instructions that implement the functions, acts, and/or operations specified in the flowcharts, sequence diagrams, and/or block diagrams. The computer program instructions may be provided to one or more processors of a general purpose computer, a special purpose computer, or other programmable data processing apparatus to produce a machine, such that the instructions, which execute via the one or more processors, cause a series of computations to be performed to implement the functions, acts, and/or operations specified in the flowcharts, sequence diagrams, and/or block diagrams.

In certain alternative embodiments, the functions, acts, and/or operations specified in the flowcharts, sequence diagrams, and/or block diagrams may be re-ordered, processed serially, and/or processed concurrently consistent with embodiments of the invention. Moreover, any of the flowcharts, sequence diagrams, and/or block diagrams may include more or fewer blocks than those illustrated consistent with embodiments of the invention.

The terminology used herein is for the purpose of describing particular embodiments only and is not intended to be limiting of the embodiments of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Furthermore, to the extent that the terms “includes”, “having”, “has”, “with”, “comprised of”, or variants thereof are used in either the detailed description or the claims, such terms are intended to be inclusive in a manner similar to the term “comprising”.

While all of the invention has been illustrated by a description of various embodiments and while these embodiments have been described in considerable detail, it is not the intention of the Applicant to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the spirit or scope of the Applicant's general inventive concept.

What is claimed is:

1. A method for tracking operation of a comfort product, the method comprising:
determining, by at least one processor, an occupancy record for the comfort product; and
providing, by the at least one processor, an indication of whether an action relating to the comfort product is recommended based on the occupancy record.

2. The method of claim 1, wherein determining an occupancy record for the comfort product comprises:
detecting a presence on the comfort product;
determining whether the presence relates to a sleep cycle; and
in response to determining that the presence relates to a sleep cycle, incrementing one or more sleep cycle counters,
wherein providing the indication of whether an action relating to the comfort product is recommended is based on the one or more sleep cycle counters.

3. The method of claim 2, wherein determining if the presence relates to a sleep cycle comprises:
determining if the presence is detected on the comfort product for a set period of time, wherein the presence relates to the sleep cycle if the presence is detected for the set period of time.

4. The method of claim 3, wherein determining if the presence is detected on the comfort product for the set period of time comprises:
(i) upon an initial detection of the presence on the comfort product, increasing a presence counter;
(ii) determining if the presence counter has reached a sleep cycle threshold value;
(iii) in response to determining that the presence counter has reached the sleep cycle threshold value:
determining that the presence relates to a sleep cycle; and
resetting the presence counter; and
(iv) in response to determining that the presence counter has not reached the sleep cycle threshold value:
determining whether a continued presence is detected on the comfort product after a set interval; and
in response to determining that the continued presence is detected on the comfort product after the set interval, increasing the presence counter and repeating steps (ii), (iii), and (iv).

5. The method of claim 4, wherein determining if the presence is detected on the comfort product for the set period of time further comprises:
in response to determining that the continued presence is not detected on the comfort service after the set interval, determining that the presence does not relate to a sleep cycle and resetting the presence counter.

6. The method of claim 2, wherein the one or more sleep cycle counters comprises a flip counter and a rotate counter, and wherein providing the indication of whether an action related to the comfort product is recommended based on the one or more sleep cycle counters comprises:
in response to the flip counter reaching a first threshold value, indicating a recommendation to flip the comfort product; and
in response to the rotate counter reaching a second threshold value, indicating a recommendation to rotate the comfort product.

7. The method of claim 6, wherein the one or more sleep cycle counters further comprises a life cycle counter, and wherein providing the indication of whether an action related to the comfort product is recommended based on the one or more sleep cycles further comprises:
in response to the life cycle counter reaching a third threshold value, indicating a recommendation to replace the comfort product.

8. The method of claim 7, wherein the recommended action comprises at least one of the recommendation to flip the comfort product and the recommendation to rotate the comfort product, and further comprising:
automatically determining if the comfort product has been at least one of flipped and rotated;
in response to automatically determining that the comfort product has been flipped, resetting the flip counter; and
in response to automatically determining that the comfort product has been rotated, resetting the rotate counter.

9. The method of claim 2, wherein determining an occupancy record for the comfort product further comprises:
in response to receiving an initiation input, initializing the one or more sleep cycle counters at zero; and
in response to receiving a reset input after the initiation input, resetting the one or more sleep cycle counters to zero,
wherein the initiation input and the reset input include an actuation of an actuator for a first set period of time.

10. The method of claim 9, wherein providing the indication of whether an action relating to the comfort product is recommended is in response to receiving a status check input after the initiation input or the reset input, the status check input including an actuation of the actuator for a second set period of time that is less than the first set period of time.

11. The method of claim 1, wherein the at least one processor comprises a first processor and a second processor, and determining an occupancy record for the comfort product comprises:
detecting, by the first processor, a presence on the comfort product;
in response to detecting the presence, wirelessly transmitting presence data from the first processor to the second processor; and
determining, by the second processor, the occupancy record based on the presence data,
wherein providing the indication of whether an action relating to the comfort product is recommended based on the occupancy record is performed by at least the second processor.

12. The method of claim 1, wherein the comfort product is a mattress.

13. The method of claim 1, wherein the at least one processor is coupled to the comfort product, and the comfort product is configured to be placed on a foundation that supplies power to the at least one processor through an inductive coupling formed between the comfort product and the foundation when the comfort product is placed on the foundation.

14. A system for tracking operation of a mattress, the system comprising:
at least one processor; and
a memory storing instructions that, when executed by the at least one processor, cause the system to:
determine an occupancy record for the mattress; and
provide an indication of whether an action relating to the mattress is recommended based on the occupancy record.

15. The system of claim 14, wherein the instructions causing the system to determine the occupancy record for the mattress comprise instructions that cause the system to:
detect a presence on the mattress;
determine if the presence relates to a sleep cycle; and
in response to determining that the presence relates to a sleep cycle, increment one or more sleep cycle counters,
wherein the instructions causing the system to provide the indication of whether an action relating to the mattress is recommended based on the occupancy record comprise instructions that cause the system to provide the indication of whether the action relating to the mattress is recommended based on the one or more sleep cycle counters.

16. The system of claim 15, further comprising:
a housing coupled to the mattress and defining an interior space, wherein the memory and the at least one processor are positioned in the interior space of the housing.

17. The system of claim 16, further comprising:
an actuator accessible from outside the housing,
wherein the instructions causing the system to determine an occupancy record for the mattress further comprise instructions that cause the system to:
in response to receiving an initiation input, initialize the one or more sleep cycle counters at zero; and
in response to receiving a reset input after the initiation input, reset the one or more sleep cycle counters to zero,
wherein the initiation input and the reset input include an actuation of the actuator for a first set period of time.

18. The system of claim 17, wherein the instructions causing the system to provide the indication of whether an action relating to the mattress is recommended further comprises instructions that cause the system to provide the indication in response to receiving a status check input after the initiation input or the reset input, the status check input including an actuation of the actuator for a second set period of time that is less than the first period of time.

19. The system of claim 16, further comprising:
a visual indicator viewable from outside the housing,
wherein the indication of whether an action relating to the mattress is recommended is provided via the visual indicator.

20. The system of claim 19, wherein the visual indicator comprises:
a first LED and one or more second LEDs,
wherein the instructions causing the system to provide the indication of whether an action relating to the mattress is recommended further comprises instructions that cause the system to:
illuminate the first LED if an action relating to the mattress is not recommended; and
illuminate one of the one or more second LEDs if an action relating to the mattress is recommended.

21. The system of claim 20, wherein the one or more second LEDs comprise an LED relating to each of a plurality of recommended actions, the recommended actions including a recommendation to flip the mattress, a recommendation to rotate the mattress, and a recommendation to replace the mattress, and if an action relating to the mattress is recommended:
the recommended action is one of the plurality of recommended actions, and
the instructions causing the system to illuminate one of the one or more second LEDs comprise instructions that cause the system to illuminate the LED relating to the recommended action.

22. A device for tracking operation of a mattress, comprising:
a housing defining an interior;
at least one processor positioned in the interior of the housing; and
a memory positioned in the interior of the housing, the memory storing instructions that, when executed by the at least one processor, cause the device to:
determine an occupancy record for the mattress; and
provide an indication of whether an action relating to the mattress is recommended based on the occupancy record.
